(19) United States
(12) Patent Application Publication
(10) Pub. No.: US 2009/0227873 A1
(43) Pub. Date: Sep. 10, 2009

(54) DATA VISUALIZATION METHOD FOR AN ULTRASOUND IMAGING SYSTEM

(75) Inventors: Robert Mesaros, Bothell, WA (US); Ulrich Rankers, Livermore, CA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/568,072

(22) PCT Filed: Apr. 14, 2005
(86) PCT No.: PCT/IB2005/051225

§ 371 Date: Oct. 19, 2006

Related U.S. Application Data
(60) Provisional application No. 60/563,353, filed on Apr. 19, 2004.

(51) Int. Cl. A61B 8/14

(52) U.S. Cl. 600/459

(57) ABSTRACT

A method for visualizing data in an ultrasound probe system in which a display is arranged in a housing of an ultrasound probe in a position in which it is visible and the display is controlled to display data relating to a transducer of the probe and/or the probe. By providing a display on connection with the housing of the ultrasound probe, the user can view functional parameters of the transducer or transducer settings and probe settings while maintaining the transducer in a common field of view with the patient or object being examined.

The present invention relates generally to a method for visualizing data relating to an ultrasound transducer probe of an ultrasound imaging system, and more particularly, to a housing of an ultrasound transducer probe that provides a built-in or integrated dynamic display on which status information about the settings of the transducer of the probe and/or other related information pertinent to the transducer is displayed.

In an ultrasound imaging system, ultrasound transducer probes are often used for interoperative imaging of a patient's internal body parts. The probes, also referred to as an ultrasonic or ultrasound head, include a housing and a transducer arranged in the housing in a position such that ultrasound waves are emitted from an active surface of the transducer toward the body part as the housing is passed over the body part. The transducer then receives ultrasound waves reflected from the body part. A cable is connected to the transducer and passes through an opening in the housing to connect the transducer to a control unit and allow for signal transmission between the transducer and the control unit. Signals representative of the received ultrasound waves are provided by the transducer to the control unit via the cable and processed by the control unit to obtain images of the body part.

The control unit includes a display screen on which the images of the body part are visualized and is coupled to one or more user interface devices which enable adjustment of transducer settings. The display screen is typically used to display status information about the transducer settings and/or other information relating to the operation of the transducer. When a user modifies the transducer settings via the user interface devices during an ultrasound imaging procedure, the modifications are reflected by changes in the display.

A drawback of such a system is that the user must avert their gaze from the transducer housing, and thus the patient against which the transducer housing is placed, during the ultrasound imaging procedure in order to view the transducer settings and ascertain whether they are correct for the imaging procedure being performed.

Ultrasound imaging systems of the type described above exist which include several ultrasound transducer probes coupled to the same control unit. The transducer probes have different imaging characteristics and each is used for a specific imaging procedure. A problem with these systems is that it is difficult to ascertain which probe is activated. It may therefore be necessary to check each probe until the activated probe is located.

It is an object of the present invention to provide a new transducer probe and method for visualizing data for ultrasound imaging systems, such as general abdominal and obstetric imaging applications.

It is another object of the present invention to provide a new transducer probe for ultrasound imaging systems which enables the probe to be easily identified as being activated when used in conjunction with other probes and coupled to a common control unit.

It is another object of the present invention to provide a new method for visualizing data in an ultrasound transducer probe that eliminates the need for a user to avert their gaze from the transducer housing in order to view transducer settings.

It is yet another object of the present invention to provide a new housing for an ultrasound transducer probe which includes a built-in or integrated dynamic display capable of showing status information about transducer settings and/or other related information pertinent to the transducer.

It is still another object of the present invention to provide a new housing for an ultrasound transducer which provides improved ergonomics with integrated technology to display relative transducer settings.

In order to achieve these objects and others, in a method for visualizing data in an ultrasound probe system, a display is arranged in a housing of an ultrasound probe in a position in which it is visible and the display is controlled to display data relating to a transducer of the probe and/or the probe itself. By providing a display on the housing of the ultrasound probe, the user can view functional parameters of the transducer or transducer settings and probe settings while maintaining the transducer in a common field of view with the patient or object being examined. Previously, the user was required to look at a control unit coupled by a cable to the housing in order to view the transducer and probe settings.

Possible transducer and probe settings include a name or designation of the transducer or the probe, a frequency setting of the transducer, activation status of the probe, a field of view setting, an indication of the orientation of the probe along with the sector in which the probe is operational, temperature of the transducer, angle of operation of the transducer, depth of penetration of waves transmitted by the transducer, needle guide parameters and quadrant settings for scan plan orientation. In addition to functional parameters of the transducer and/or probe, the display could also be used to display data relating to a patient being examined with the probe and status of the probe during test cycles.

In one embodiment, the display is controlled to be illuminated differently depending on the activation status of the probe. For example, the display is placed in a blank state when the probe is inactive, a name or designation of the transducer or the probe is displayed when the probe receives power but is not operative, and the display is backlit when the probe is operative. Thus, a user can readily ascertain the status of availability of the probe simply by viewing the display.

When a plurality of such probes are included in an ultrasound probe system having a control unit and one or more user interfaces coupled to the control unit, the user can readily ascertain the status of each probe and determine which probe or probes are ready for use. This improves workflow efficiency and productivity.

The invention, together with further objects and advantages thereof, may best be understood by reference to the following description taken in conjunction with the accompanying drawings, wherein like reference numerals identify like elements and wherein:

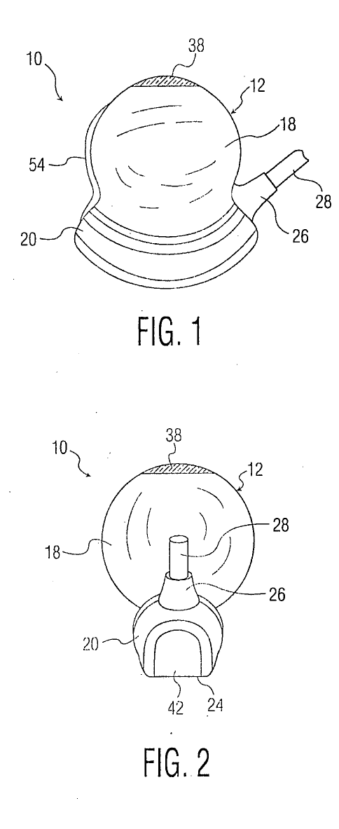

FIG. 1 is a front view of an ultrasound transducer probe in accordance with the present invention.

FIG. 2 is a side view of the probe shown in FIG. 1.

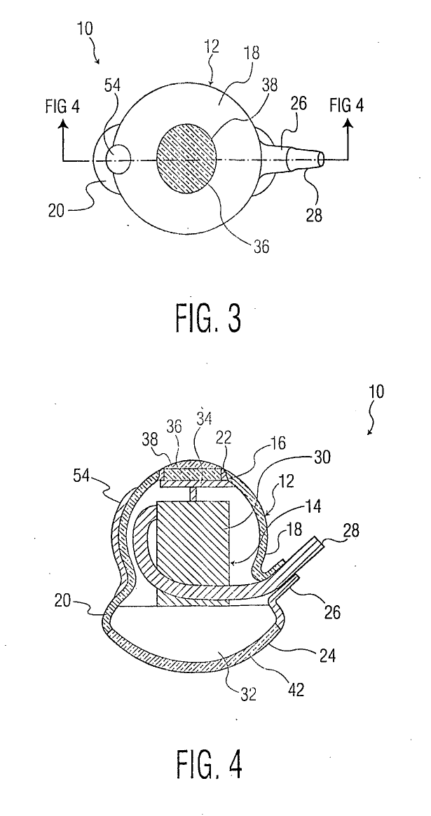

FIG. 3 is a top view of the probe shown in FIG. 1.

FIG. 4 is a cross-sectional view taken along the line 4-4 of FIG. 3.

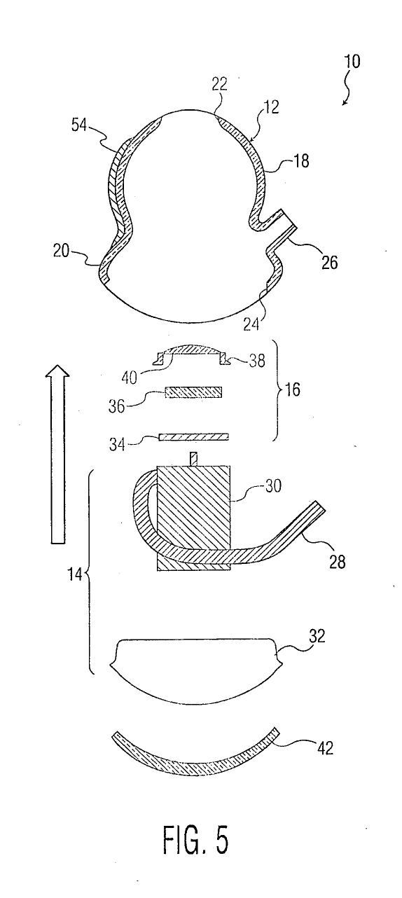

FIG. 5 is an exploded view of the probe shown in FIG. 1.

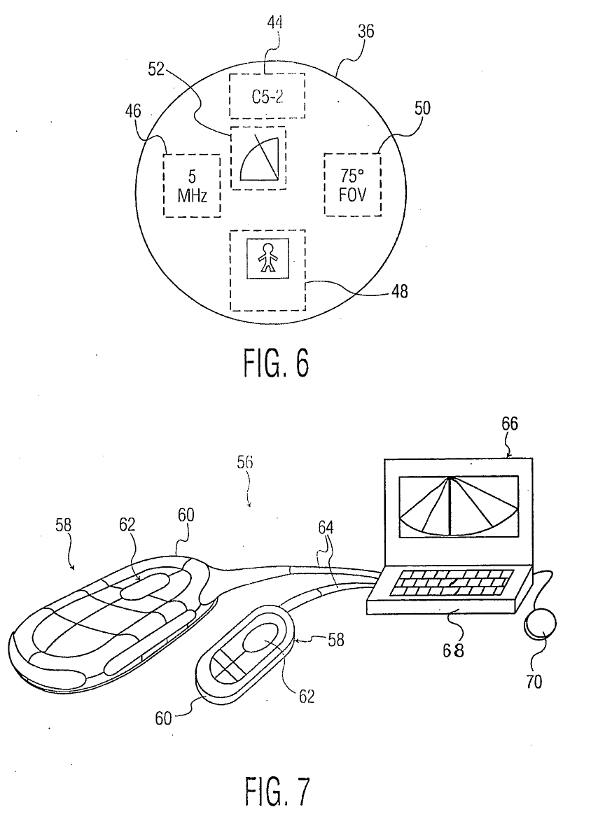

FIG. 6 is an enlarged view of the display screen of the probe shown in FIG. 1.

FIG. 7 is a perspective view of another embodiment of an ultrasound transducer probe in accordance with the invention.

Referring to the accompanying drawings wherein like reference numerals refer to the same or similar elements, FIGS. 1-5 show an ultrasound transducer probe in accordance with the invention which is designated generally as 10. The probe 10 includes a housing 12, a transducer 14 arranged in the housing 12 and a display unit 16 arranged in the housing 12. Display unit 16 may be built in or integrated into the housing 12.

Housing 12 has an upper portion 18 having a substantially spherical outer surface and a lower portion 20 having the approximate, general shape of a truncated cone. The upper portion 18 is designed to be easily grippable by a user and includes an opening 22 at a top part thereof. The lower portion 20 includes an opening 24 through which ultrasonic waves are transmitted and received by the transducer 14 during use of the probe 10. The lower portion 20 also includes a cable conduit 26 through which a cable 28 passes to connect the transducer 14 to a control unit (not shown). Optionally, the probe 10 is weighted to balance it in its neutral position by, e.g., filling a part or all of the available interior of the housing 12 with a loaded, flexible epoxy, or similar material.

Cable conduit 26 is designed to lead or orient the cable 28 out of the housing 10 in a particular direction, preferably at an angle of about 0° to about 30° relative to a horizontal plane of the housing 12 perpendicular to a central, vertical axis thereof. By positioning an exit opening of the cable conduit 26 below the top portion 18 of the housing 12 which is gripped by the user, in combination with the specific orientation of the cable conduit 26 at an angle to the horizontal plane of the housing 12, minimizes the effective weight/torque which arises during movement of the housing 12 and provides improved ergonomics for the user. Additional details relating to the construction of the housing 12 and ergonomic advantages obtained thereby are set forth in U.S. provisional patent application Ser. No. 60/526,818 filed Dec. 4, 2003 entitled “Ergonomic Housing For Ultrasound Transducer Probe” incorporated by reference herein.

Transducer 14 may be any type of transducer capable of performing imaging. As shown, transducer 14 includes an electronic imaging unit 30 connected to the cable 28 and a crystal/acoustic component assembly 32 which cooperates with the imaging unit 30 to transmit ultrasonic waves having the necessary properties to enable the formation of images from waves reflected by the object being examined. The imaging unit 30 may include a printed circuit board having the necessary electronic components to transmit, receive and process ultrasonic waves. Optionally, an EMI-RFI shield layer may be provided around the transducer 14.

The display unit 16 is arranged above the transducer 14 and includes a display driver unit 34, a LCD display 36 connected thereto and a display cover 38 arranged in the opening 22 of the upper portion 18 of the housing 12 and above the display 36. An underside of the display cover 38 includes a recess 40 which receives the display 36 (see FIG. 5). The display cover 38 is made of a transparent material to enable visualization of the display 36 therethrough. Alternatively, the housing 12 can be made of a transparent material with the display 36 arranged therein, in virtually any location, and visible through the transparent housing 12. In this embodiment, an open window would not have to be formed in the housing 12 to receive the display cover 40. Also, only part of the housing 12 opposite the display 36 would have to be made of a transparent material and this can be achieved using a process called in-mold decorating which enables the selective positioning of a clear area for viewing of the display 36.

Display driver unit 34 may comprise a printed circuit board on which the necessary components for forming signals to control the display 36 are mounted. Display driver unit 34 is electrically coupled to the imaging unit 30 and receives power and control signals from the control unit via the imaging unit 30.

The display unit 16 is constructed to provide a selectively illuminated display area on display 36 depending on the activation status of the probe 10. For example, in one embodiment, when the probe is inactive, i.e., the probe is disconnected from the control unit or off, the display area of display 36 will be blank without any illumination. When the probe 10 receives power from the control unit via cable 28, a name or designation of the probe 10 will be outlined indicating that the probe 10 is ready for use and when the probe 10 is powered on and operating, the display 36 will be backlit. The display 36 can either be independently programmed to accomplish the selective illumination, e.g., via display driver unit 34, or via the control unit when the probe 10 is coupled thereto.

As such, when a plurality of similar probes 10 in accordance with the invention are connected to the same control unit, it can be readily ascertained which probe is activated because the display 36 of the activated probe 10 will be operative and the name or designation of the transducer or probe will be shown. The user can therefore reach immediately for the activated probe instead of handling each probe from among the plurality of similar probes to ascertain whether it is the activated probe. This saves time during ultrasound examinations and improves productivity and efficiency. Moreover, when switching between different probes, i.e., deactivating one probe and activating another probe, the newly activated probe can be readily picked up for immediate use.

Another advantage arising from the presence of display unit 16 on the housing 12 is the ability to display information relating to the transducer 14 on the display 36. Specifically, in addition to the name or designation of the transducer or probe, settings of the transducer 14 can be shown on display 36 so that the user does not have to avert their gaze from the housing 12 which would typically be placed against the patient being examined. Since the user can maintain their view of the examining area of the patient while simultaneously viewing transducer settings, workflow efficiency is improved. The display of the transducer settings in display 36 would be instead of or in addition to the display of such transducer settings on a screen of the control unit.

Referring now to FIG. 6, the transducer settings which can be displayed on display 36 during an examination procedure are key functional parameters including, but not limited to, the transducer name or designation (in display area 44), the frequency settings (in display area 46), activation status such as inactive, ready, active (in display area 48 with an icon indicative of the status), field of view (FOV) setting (in display area 50), orientation of the probe along with the sector in which the probe is operational (in display area 52), temperature, angle, depth, needle guide parameters and quadrant settings for scan plan orientation. The display 36 can be designed to display data over its entire active surface with the different parameters being displayed in designated portions of the active surface area such as shown in FIG. 6. The location and size of the designated display areas 44, 46, 48, 50, 52, and the content thereof, can be determined as desired.

The specific parameters to be displayed on the display 36 can be programmed via the control unit and user interface devices coupled thereto. The parameters can thus be varied for different probes and for different examination procedures as desired.

Display of parameters relating to the transducer 14 is primarily useful prior to the beginning of the actual ultrasound examination of the patient. That is, prior to placing the probe 10 against a patient, the settings of the transducer 14 must be within a predetermined operating range to enable the generation of useful ultrasound images.

The display 36 can also be programmed to display data relating to the patient being examined. The display 36 can also be used to communicate the status of the ultrasound probe 10 to the user during test cycles. However, the display 36 is not used to display the images being formed from the returned ultrasonic waves received by the transducer 14. The display driver unit 34 therefore receives signals indicative of parameters relating to the transducer 14, and optionally the patient, and does not receive any video display signals

The probe 10 also includes a lens 42 arranged in the opening 24 in the lower portion 22 of the housing 12 and through which ultrasonic waves pass to and from the active surface of the transducer 14 (see FIG. 4). Lens 42 is attached to the housing 12 by any means known in the art, such as by an adhesive agent. In the alternative, the lens 42 may be attached to or formed in connection with the transducer 14.

In addition, an orientation marker 54 is connected to the housing 12 to enable the orientation of the housing 12 to be determined relative to a predetermined or designated reference line and displayed on the display 36.

To assemble the probe 10, the electronic imaging unit 30 is connected to cable 28 and the display driver unit 34 is connected to the electronic imaging unit 30. The display 36 is connected to the display driver unit 34 and the crystal/acoustic component assembly 32 is connected to the electronic imaging unit 30. The display cover 38 is arranged over the display 36. Any of the foregoing components might be formed integral with one another so that separate assembly step would not be required to form this subassembly. The housing 12 may be formed as a two part housing 12 so that the subassembly is mounted into one housing component with the cable 28 passing through the cable conduit 26 and then the housing components are attached to one another with the subassembly and the lens 42 therebetween. If the lens 42 is part of the housing 12, then it would be first attached to one of the housing components and then to the other housing component when the housing components are attached to one another. Alternately, the lens 42 could be part of or formed integral with the transducer 14.

With the foregoing structure, the housing 12 with the integrated display unit 16 dynamically provides key status information about the transducer settings and/or relative system settings to the user at any time while the system is powered on and the transducer 14 is connected to the control unit.

Another embodiment of an ultrasound probe system including ultrasound transducer probes in accordance with the invention is shown in FIG. 7 and designated as 56. The system 56 includes a plurality of similar probes 58 with each probe 58 including a housing 60 and an integrated display unit which includes a display 62 visible through a transparent display cover thereof, or through a transparent portion of the housing 60. Housing 60 includes a transducer in an interior thereof which is connected via a cable 64 to a control unit 66 which includes a microprocessor. User interface devices 68, 70 are connected to the control unit 66. The display unit may be programmed to display on the display 62 any of data discussed above with respect to display unit 16 and display 36 thereof, e.g., transducer settings and patient data. Probes 58 may have the shape as shown in FIG. 7 or the shape of the probe 10 shown in FIGS. 1-5 above, with the same structure thereof.

Control unit 66 controls the display units, when the probes 58 are coupled via the cable 64 thereto, to vary activation and content of the display 62 based on the activation status of the probe 58 and the desired functional parameters of the transducer and/or probe 58. In the absence of a connection between the probes 58 and the control unit 66, the display 62 can be designed to maintain a blank, non-illuminated screen which would be indicative of an inactive status.

Although only two housing constructions are illustrated, the invention can be applied to any shape and size of an ultrasound probe housing. Applying teachings of the invention, the probe housing would be constructed to include an integral display and programmed to display data relating to the transducer arranged in the housing and/or the patient being examined by the probe.

Although illustrative embodiments of the present invention have been described herein with reference to the accompanying drawings, it is to be understood that the invention is not limited to these precise embodiments, and that various other changes and modifications may be effected therein by one of ordinary skill in the art without departing from the scope or spirit of the invention.

1. A method for visualizing data in an ultrasound probe system, comprising the steps of:
arranging a transducer in a housing of an ultrasound probe;
arranging a display in the housing in a position in which the display is visible; and
controlling the display to display data relating to at least one of the transducer and the probe.

2. The method of claim 1, wherein the step of arranging the display in the housing comprises integrating the display into the housing.

3. The method of claim 1, wherein the step of arranging the display in the housing comprises arranging the display above the transducer and such that the display is visible through an opening in an upper surface of the housing.

4. The method of claim 1, further comprising the steps of:
forming an opening in an upper surface of the housing; and
arranging a transparent cover in the opening, the display being arranged in alignment with the cover.

5. The method of claim 1, further comprising the steps of:
coupling the display to the transducer; and
providing power and control signals to the display via the transducer.

6. The method of claim 1, further comprising the step of controlling the display to be illuminated differently depending on activation status of the probe.

7. The method of claim 6, wherein the step of controlling the display comprises placing the display in a blank state when the probe is inactive, outlining a name or designation of the transducer or the probe on the display when the probe receives power but is not operative, and backlighting the display when the probe is operative.

8. The method of claim 1, wherein the step of controlling the display comprises controlling the display to display functional parameters of the transducer for an ultrasound examination procedure at least prior to initiation of the procedure.

9. The method of claim 1, wherein the step of controlling the display comprises controlling the display to display a name or designation of the transducer or the probe.

10. The method of claim 1, wherein the step of controlling the display comprises controlling the display to display a frequency setting of the transducer.

11. The method of claim 1, wherein the step of controlling the display comprises controlling the display to display activation status of the probe.

12. The method of claim 1, wherein the step of controlling the display comprises controlling the display to display a field of view setting.

13. The method of claim 1, wherein the step of controlling the display comprises controlling the display to display an indication of the orientation of the probe along with the sector in which the probe is operational.

14. The method of claim 1, wherein the step of controlling the display comprises controlling the display to display at least one of temperature of the transducer, angle of operation of the transducer, depth of penetration of waves transmitted by the transducer, needle guide parameters and quadrant settings for scan plan orientation.

15. The method of claim 1, wherein the step of controlling the display comprises controlling the display to a plurality of parameters selected from a group consisting of a name or designation of the transducer or the probe, a frequency setting of the transducer, activation status of the probe, a field of view setting, an indication of the orientation of the probe along with the sector in which the probe is operational, temperature of the transducer, angle of operation of the transducer, depth of penetration of waves transmitted by the transducer, needle guide parameters and quadrant settings for scan plan orientation.

16. The method of claim 1, further comprising the step of controlling the display to display data relating to a patient being examined with the probe.

17. The method of claim 1, further comprising the step of controlling the display to display status of the probe during test cycles.

18. An ultrasound probe system, comprising:
a control unit;
at least one user interface coupled to said control unit; and
a plurality of probes connected to said control unit,
each of said probes including a housing, a transducer arranged in said housing and having an active surface oriented toward a bottom of said housing, and a display arranged in said housing, said housing being arranged to provide a clear view of said display, said control unit being arranged to control said display to vary activation and content of said display based on activation status of said probe.

19. The system of claim 18, wherein said display of each of said probes is arranged to be illuminated or refrain from illumination depending on activation status of said probe.

20. The system of claim 18, wherein said display of each of said probes is arranged to be in a blank state when said probe is inactive, outline a name or designation of said transducer or said probe when said probe receives power but is not operative, and to be backlit when said probe is operative.
